(19) United States
(12) Patent Application Publication
(10) Pub. No.: US 2008/0033443 A1
(43) Pub. Date: Feb. 7, 2008

(54) System and Method for Tissue Resection

(75) Inventors: George Sikora, Bridgewater, MA (US); Steven W. Ek, Bolton, MA (US); Tim Brightman, Franklin, MA (US)

Correspondence Address: GROSSMAN, TUCKER, PERREAULT & PFLEGER, PLLC, 55 SOUTH COMMERICAL STREET, MANCHESTER, NH, 03101, US

(73) Assignee: ARTHROSURFACE INCORPORATED, Franklin, MA (US)

(21) Appl. No.: 11/779,044

(22) Filed: Jul. 17, 2007

Related U.S. Application Data
(60) Provisional application No. 60/807,538, filed on Jul. 17, 2006.

(51) Int. Cl. A61B 17/16

(52) U.S. Cl. 606/84; 606/87

(57) ABSTRACT

The present disclosure describes an apparatus for tissue resection. At least one embodiment described herein provides a tissue resection system that includes a cutter that includes a blade system and a guide bar. The blade system is configured to be slidably coupled to said guide bar to permit said blade system to move relative to the guide bar, and said blade system comprises a chain of a plurality of links hingedly coupled together, wherein at least one said link comprising a cutting portion to cut tissue. The blade system has a thickness configured to generate a recessed pocket in the tissue. Of course, many alternatives, variations and modifications are possible without departing from this disclosure.

This application claims the benefit under 35 USC 119(e) of U.S. Provisional Application Ser. No. 60/807,538, filed Jul. 17, 2006, the teachings of which are hereby incorporated by reference in their entirety.

### FIELD

The present disclosure describes a system and method for resecting tissue, for example, to create an implant site for an orthopedic repair component.

### BACKGROUND

One approach to repairing a damaged articular joint may involve resecting at least a portion of one or more damaged articular surfaces so that they may receive a repair component that may replace a portion, or all, of the articular joint. The repair component may be a prosthetic or a biological repair. Various tools and systems have been employed for resecting bone to create an implant site having a desired size and shape that may receive the repair component. Cutting tools, such as blade saws, burrs, osteotomes, and rotary drills may be used for resecting the desired portions of bone. These cutting tools may be used in conjunction with associated guides and templates, to create the desired implant sites.

Creating relatively complicated implant sites may often require multiple cuts, which may require a variety of angles of approach. The conventional cutting tools may have limitations in terms of geometric capability. The various cuts that may be made to achieve a final desired implant site are properly aligned with respect to relevant anatomical structures, as well as with respect to the other cuts. Multiple cuts from varying angles of approach may require a larger access incision, or multiple large access incisions. Additionally, implementation and coordination of the various cutting guides and instruments may often be extremely complicated, requiring a high level of skill in the clinician and increasing the chance of error.

### BRIEF DESCRIPTION OF DRAWINGS

Features and advantages of the claimed subject matter will be apparent from the following detailed description of exemplary embodiments consistent therewith, which description should be considered with reference to the accompanying exemplary drawings, wherein:

FIG. 1 schematically depicts an exemplary embodiment of a cutter consistent with the present disclosure;

FIG. 2 schematically depicts an exemplary embodiment of a cutting guide consistent with the present disclosure;

FIG. 3 shows another exemplary embodiment of a cutter and a cutting guide consistent with the present disclosure;

FIG. 4 shows the exemplary cutter of FIG. 3 including an exemplary drill as an external power source;

FIG. 5 is a close-up view showing the coupling between the drill and the exemplary cutter of FIG. 4;

FIG. 6 depicts the exemplary cutter of FIG. 3 in exemplary operation resecting a portion of a model of a tibial articular surface and adjacent bone;

FIG. 7 is another view of the exemplary cutter of FIG. 3 in exemplary operation resecting a portion of a model of a tibial articular surface and adjacent bone;

FIG. 8 depicts the exemplary resected slot created in the model of the tibia using the exemplary cutter of FIG. 3;

FIG. 9 depicts an exemplary embodiment of a positioning system which may be used in connection with a resection system according to the present disclosure;

FIGS. 10 and 11 depict the use of exemplary guide pins that may be used to establish an orientation and position relative to tissue to be resected;

FIGS. 12 through 16 depict an exemplary cutting guide and the exemplary use thereof for resecting tissue; and

FIG. 17 depicts an exemplary resected pocket created according to an exemplary embodiment of the system of the present disclosure.

### DETAILED DESCRIPTION

Generally, this disclosure describes a system and method that may be used for resecting tissue, for example, bone, cartilage, or other tissue. While this disclosure is generally set forth in the context of resecting an articular surface and adjacent bone, e.g., to create a pocket or recess for receiving a repair component implant, such as a prosthetic articular surface implant or biological repair, it should be understood that the system and method herein may be applicable to resecting any bone, cartilage, or other tissue, and should not be limited to the context of creating an implant site for a repair component replacing at least a portion of an articular surface.

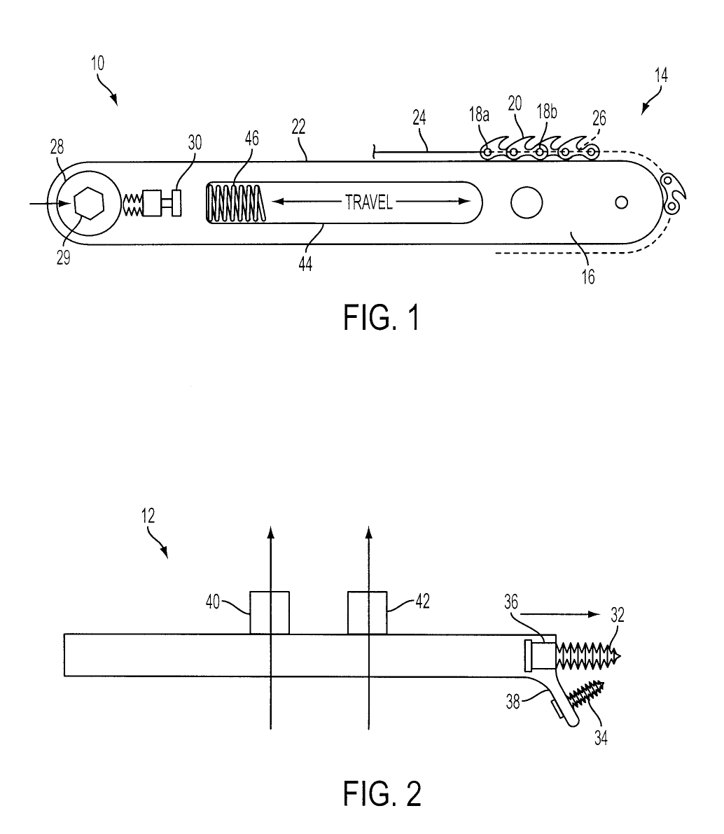

FIGS. 1 and 2 depict an exemplary tissue resection system according to one embodiment of this disclosure. The system may include a cutter 10 (FIG. 1) and an exemplary cutting guide 12 (FIG. 2). The cutting guide 12 may be removably secured relative to an anatomical feature. The cutter 10 may be slidably coupled to the cutting guide 12 to orient the cutter 10 with respect to tissue to be resected. Generally, the cutter 10 may be introduced along a path into an articular surface and/or surrounding bone or other tissue, and may be configured to resect or mill a slot in the articular surface and/or the surrounding or adjacent bone and/or other tissue. The path of the cutter 10 may be controlled, at least in part, by the cutting guide 12. For example, the cutting guide 12 may control the position, orientation, and depth of the slot that may be created (milled) in the articular surface and/or bone or tissue.

As shown in FIG. 1, the cutter 10 may include a blade system 14 for removing articular cartilage, bone, and/or other tissue, and a guide bar 16 at least partially supporting the blade system 14. The blade system 14 may be slidably coupled to the guide bar 16 to permit the blade system 14 to move relative to the guide bar 16. The blade system 14 may include a plurality of links, e.g., 18a, 18b, with each link 18a, 18b being coupled to adjacent links to form an endless loop that may be flexible in at least one plane (to permit, for example, the links to rotate about the guide 16). For example, each of the plurality of links 18a, 18b may be hingedly coupled to adjacent links having parallel hinge axes. At least a portion of the plurality of links 18a, 18b may include an outwardly facing cutting portion 20 to cut the tissue to be resected. The cutting portions 20 may include one or more teeth, which may have sharpened and/or abrasive portions. According to an embodiment, the blade system 14 may be generally reminiscent of aspects of a chain saw and/or mortise saw chain. Alternatively, the blade system 14 may include an endless loop of flexible material, e.g., rubber, fabric, metal, etc., such as, for example, a belt, which may include at least one cutting portion 20.

The guide bar 16 may support the blade system 14 around at least a portion of the periphery 22 of the guide bar 16. For example, the guide bar 16 may include a protruding rib 24 disposed around at least a portion of the periphery of the guide bar 16. At least a portion of the rib 24 may be received in a complementary groove 26, indicated by broken line, or recess in at least one link 18a, 18b of the blade system 14. In other embodiments, the guide bar 16 may include a peripheral groove or channel extending around at least a portion of the guide bar 16. At least a portion of the blade system 14, e.g., at least a portion of one or more links 18a, 18b, including the link itself or a protrusion or portion thereof, may be at least partially disposed in the groove.

The cutter 10 may include a drive mechanism 28 to drive the blade system 14 to travel around the perimeter of the guide bar 16. The drive mechanism 28 may include a sprocket, drive wheel, etc., configured to engage at least a portion of the plurality of links 18a, 18b. In one embodiment, the drive mechanism 28 may be configured for rotation about an axis generally perpendicular to a plane of the guide bar 16. A drive axle 29 may be associated with the drive mechanism 28 so that an external power source, such as a drive motor, hand drill, etc., may be coupled to and may rotatably drive the drive mechanism 28. Alternatively, the cutter 10 may include an integral power source for rotating the drive to drive the blade system 14 around the perimeter of the guide bar 16.

In other embodiments, the blade system 14 may not be formed as an endless loop traveling around the guide bar 16. For example, the blade system 14 may include a flexible member having respective opposed ends and including at least one cutting portion 20. The flexible member may be disposed around at least a portion of the perimeter of the guide bar 16 and may be configured for oscillating, or back-and-forth, movement around at least a portion of the perimeter of the guide bar 16, rather than movement in only a single direction. Of course, a blade system configured as an endless loop may also be susceptible to an oscillating mode of operation. Various other configurations may also be suitably employed in connection with a cutter 10 of the present disclosure. The cutter 10 may also include, for example, a tensioning screw 30 for controlling the tension of the blade system 14, a lubricating systems (not shown), a gear train (not shown—e.g., associated with the drive portion 28, etc.).

As shown in FIG. 2, the cutting guide 12 may include a longitudinal member 39 and a locating member 38. The locating member 38 may be disposed at an angle relative to the longitudinal member 39, to provide orientation of the longitudinal member 39 with respect to tissue to be resected. To this end, the cutting guide 12 may be configured to be positioned relative to an articular surface, or tissue, to be resected and to constrain or control the movement of the cutter 10 relative to the articular surface, or tissue, to be resected. In this embodiment, the cutting guide 12 may be oriented relative to the articular surface, or bone/tissue, to be resected using the screws 32, 34 to maintain the cutting guide 12 in selected orientation. The cutting guide 12 may be removably affixed relative to the articular surface, or bone, using at least one screw 32 and/or 34 that may be coupled to the locating member 38.

The cutting guide 12 may include one or more guide portions configured to control the movement of the cutter 10 relative to the cutting guide 12. As shown in FIG. 2, the guide portions may include at least one protrusion, such as bosses 40, 42, that may be positioned along the longitudinal member 39. The bosses 40, 42 may be configured to be at least partially received in a complementary slot 44 defined in the guide bar 16 and may slidably couple the guide bar 16 to the longitudinal member 39. The interaction of the bosses 40, 42 of the cutting guide 12 and the slot 44 in the guide bar 16 may control the path of the cutter 10 and the depth of the resection. For example, when the two bosses 40, 42 are at least partially received in the slot 44, the trajectory, or path of movement, of the cutter 10 may be constrained to an axis of the slot 44. Similarly, the travel of the cutter 10, i.e., the depth of resection, may be constrained by the length of the slot 44 relative to the spacing of the bosses 40, 42. The length of the slot 44 relative to the spacing of the bosses 40, 42 may be adjustable to allow the depth of resection to be varied. For example, an adjustable obstruction, e.g., a screw, may be disposed relative to the slot 44 to control the travel of the cutter 10 relative to the cutting guide 12. Additionally, the cutter 10 may be biased toward a retracted position, i.e., a position away from the tissue to be resected, e.g., by a spring 46 disposed in the slot 44.

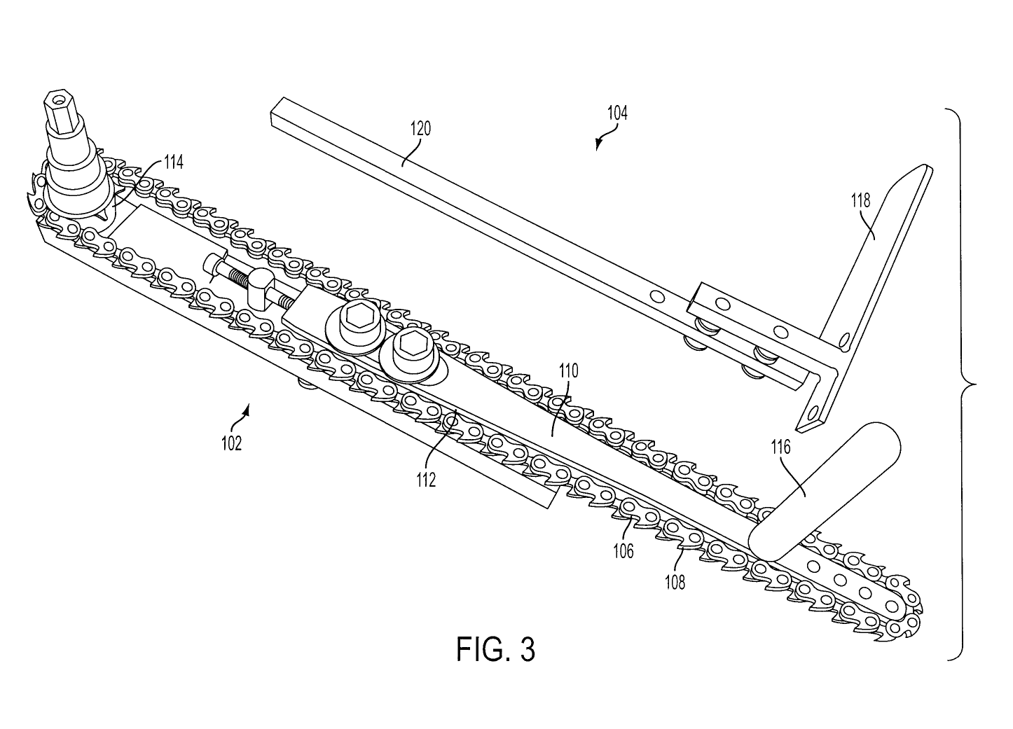

FIGS. 3-8 depict another exemplary embodiment of a resection system consistent with the present disclosure. Similar to the previous embodiment, the cutter 102 may generally include a blade system that may be the form of a linked cutting chain 106 in which at least a portion of the links may include teeth 108. The blade system may be configured to be slidably coupled to the guide bar 110 to permit the blade system to move relative to the guide bar 110. The cutting chain 106 may be disposed around at least a portion of a guide bar 110. The guide bar 110 may include a peripherally extending rib 112 which may be received in a cooperating groove defined in at least a portion of the links that may make up the cutting chain 106. The cutting chain 106 may be driven around the guide bar 110 by a drive sprocket 114, which may engage the cutting chain 106. The cutter 102 may further include a base member 121 that may be coupled to the guide bar 110. The cutter 102 may further include a handle 116, which may facilitate moving the cutter 102, e.g., for advancing the cutter 102 into tissue to be resected, such as cartilage, bone or other tissue.

The cutting guide 104 may include an alignment portion such as a longitudinal member 120 and may further include a locating member 118 that may be disposed at an angle to the longitudinal member 120. The locating member 118 may be used to orient the longitudinal member 120 relative to tissue to be resected. According to an embodiment, the cutting guide 104 may be removably secured to a bone, e.g., a portion of which is to be resected. For example, the locating member 118 may be clamped, or temporarily screwed, to the bone being resected. The longitudinal member 120, which may be directly or indirectly coupled to the cutter 102, may, at least in part, guide the movement of the cutter 102 relative to the tissue to be resected. For example, the longitudinal member 120 may be slidably coupled to the base member 121 to position the cutter 102 relative to the tissue to be resected.

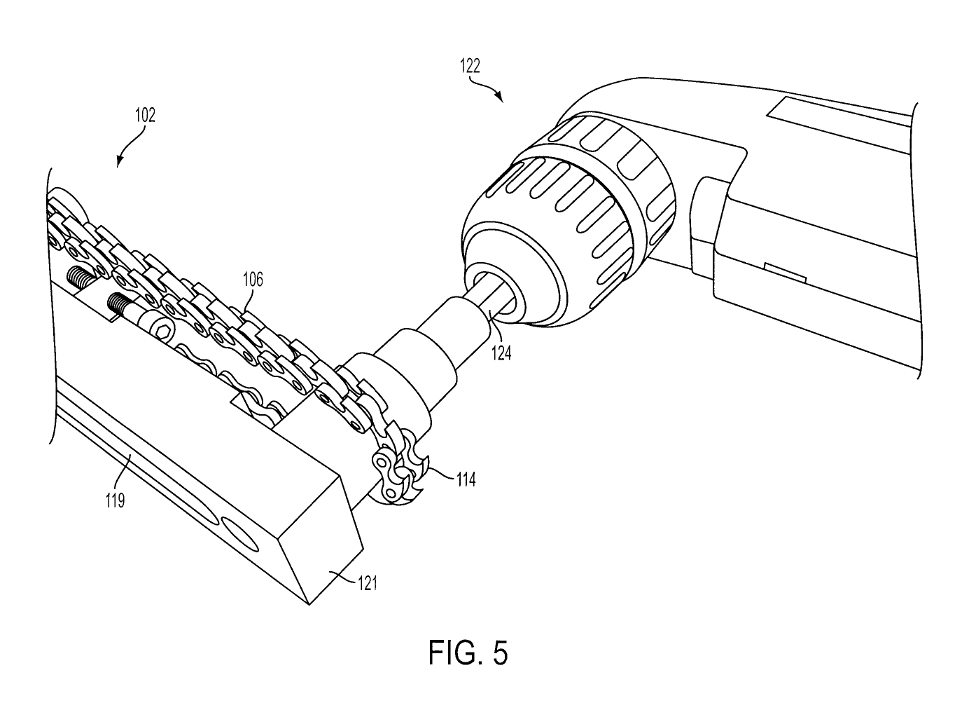

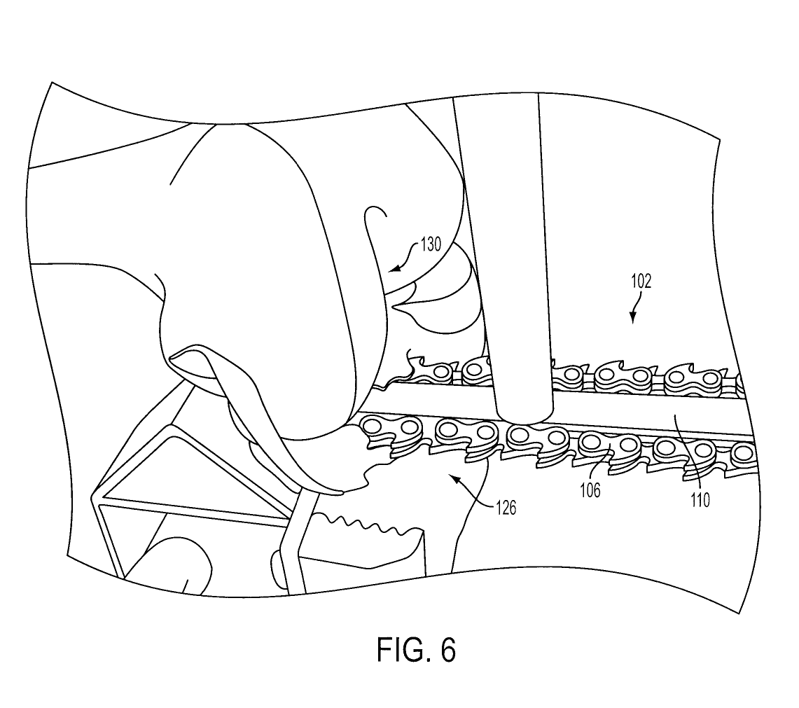

In an embodiment, the cutter 102 may be slidably coupled to the cutting guide 104, e.g., via the longitudinal member 120. For example, the longitudinal member 120 may be at least partially received in a slot, e.g., slot 119 in FIG. 5, or other aspect, of a base member of the cutter 102, e.g., base member 121 of FIG. 5. In such an embodiment, the cutter 104 may travel along the path, or trajectory, defined by the longitudinal member 120. The cutter 102 may be restrained against movement other than axial movement along the axis defined by the longitudinal member 120. For example, the cutter 102 may be prevented from one or more of moving transversely, rotating, or yawing relative to the longitudinal member 120. As used herein, rotating relative to the longitudinal member 120 may be understood to mean rotation about the axis defined by the longitudinal member 120, and yawing may be understood to mean rotation in any plane that contains the axis defined by the longitudinal member 120. In other embodiments, the cutter 102 may be provided with greater freedom of movement than simple axial displacement along the axis of the longitudinal member 120. For example, the cutter 102 may be permitted to experience at least some degree of transverse translation, rotation, and/or yaw. In related embodiments, the greater freedom of movement may be, at least in part, restricted to a defined range of movement.

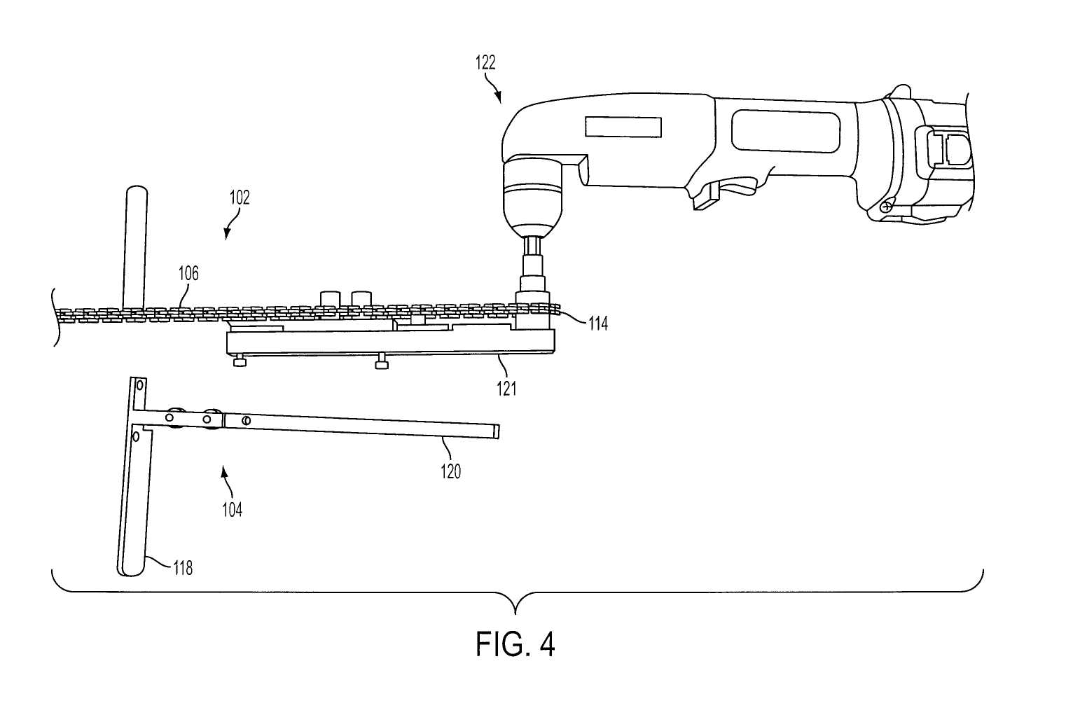

As shown, for example, in FIGS. 4 and 5, the cutting chain 106 of the cutter 102 may be driven by an external power source, such as an electric motor, e.g., in the form of a drill 122. As shown, the drill 122 may be coupled to the sprocket 114 by inserting a drive axle 124 into the drill chuck. The drill 122 may rotatably drive the sprocket 114, and thereby drive the cutting chain 106 around the guide bar 110. The cutter 102 may, in this manner, be powered by a drill 122, which may be commonly available in a clinical environment, thereby reducing the size, weight, and complexity of the cutter 102. Other external power sources, e.g., pneumatic, hydraulic, etc., may also suitably be employed for driving the cutting chain 106. Furthermore, the power source may be provided as an integrated portion of the cutter assembly, rather than as a separate element.

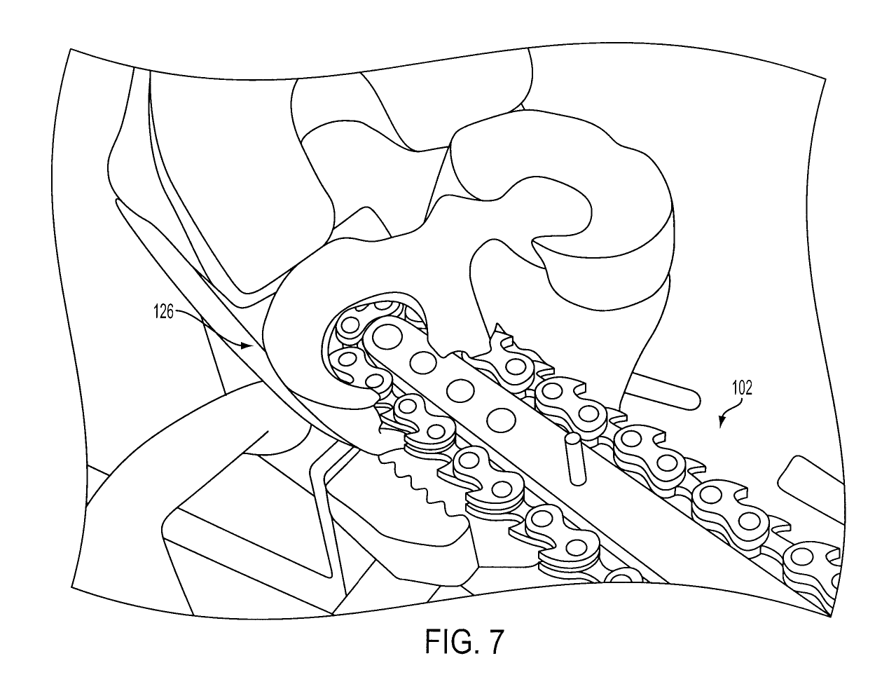

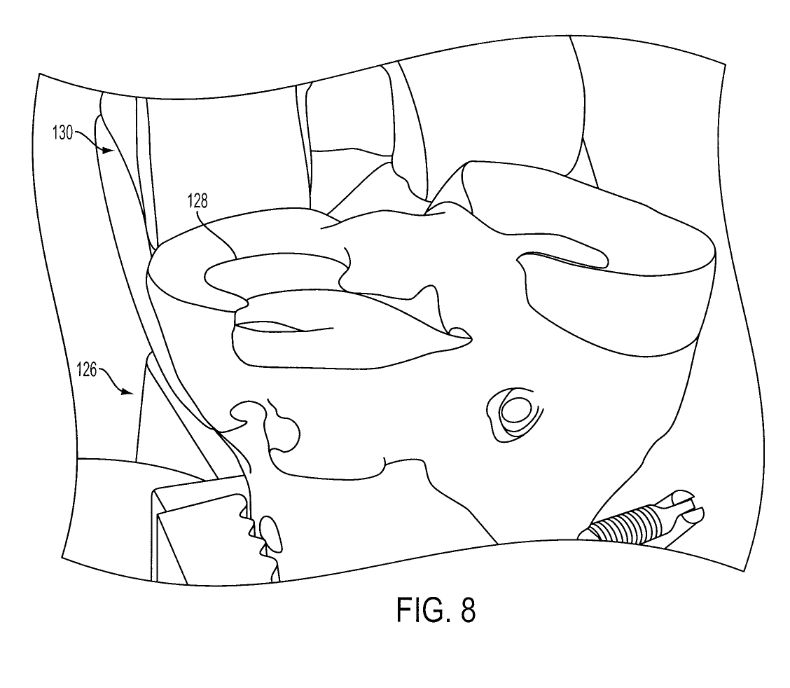

As shown, for example, in FIGS. 6 and 7, tissue 126, such as a portion of a tibial articular surface and/or adjacent bone or other tissue, may be resected by advancing the cutter 102 into the tissue 126 as the cutting chain 106 may be driven around the guide bar 110 to provide a continuous cutting action around the perimeter of the cutter 102. The cutter 102 may be advanced into the tissue 126 resecting the tissue 126 along the path of the cutter 102. While not shown, as previously discussed the path of the cutter 102 may be controlled by the cutting guide. Similarly, the cutting guide 110 may be configured to control the depth of penetration of the cutter 102 into the tissue 126.

As shown in FIG. 6, placement and trajectory of the cutter 102 may be controlled so that a portion of tissue 126 may be resected while preserving neighboring structures. For example, as shown, the cutter 102 may resect a portion of a tibia, which may include a portion of the tibial articular surface, without damaging an adjacent feature, e.g., a femoral condyle 130. Similarly, resection of a portion of the tibia may also be accomplished without damaging other adjacent structures, such as the anterior cruciate ligament or the meniscus, thereby minimizing the collateral damage. This may minimize, or eliminate, the need to make additional incisions to protect adjacent nervo-vascular structures. Furthermore, the resection of a portion of the tibial articular surface may be accomplished with minimal, or no, dislocation of the joint. Of course, as with all aspects of the present disclosure, this aspect may be similarly applicable to other parts of the anatomy.

As shown, for example, in FIG. 8, the cutter 102 may create a resected slot 128 having a specific shape and volume. A resected pocket may generally have the shape of a slot bounded by a full radius at the closed end of the slot. The rounded geometric shape of the resected slot may reduce the occurrence of stress concentration, as may result from a hard angle. Additional cutting, grinding, etc., operations may be employed to remove any overhanging margins around the slot, etc., or to further alter the resected slot.

The size and shape of the slot 128 may be based, at least in part, on the size and geometry of the cutter 102. For example, the height and geometry of the resected pocket may be based, at least in part, on the height, e.g., thickness, of the cutting chain 106, the geometry of the guide bar 110, configuration of the cutting chain 106, e.g., link pitch, tooth pattern, etc., as well as the cut trajectory and depth. More than one intersecting, or at least partially overlapping, cutting passes may be employed to achieve resected pockets having still other heights and geometries. The forgoing attributes of the cutter may be chosen to facilitate creating resected pockets having specific or general desired characteristics.

A resected slot may be created using a single cut along a single trajectory. As such, the procedure may be performed with reduced access to the resection site. The resected slot may be created from a single access point without exposing a substantial portion of the tibial surface. A procedure consistent with the foregoing may generally be considered less invasive than traditional procedures. Depending upon the size parameters of the cutter, a desired resection may be accomplished via incisions as small as about two inches, although this should not be construed as limiting the present disclosure.

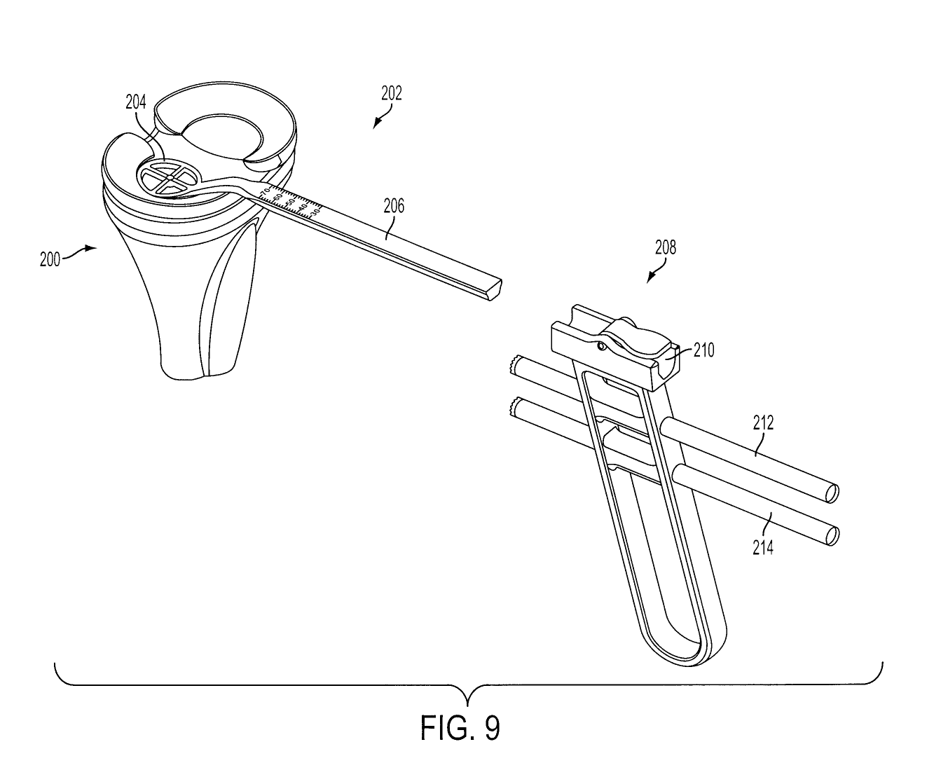

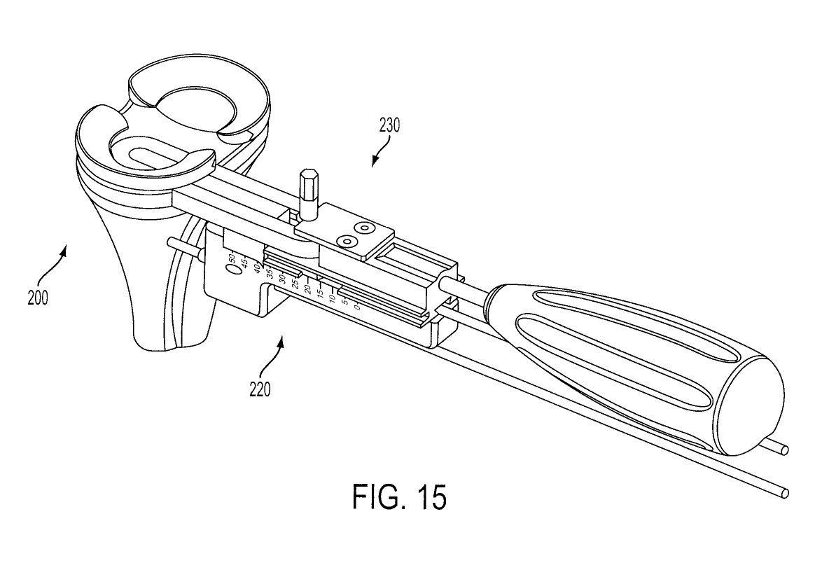

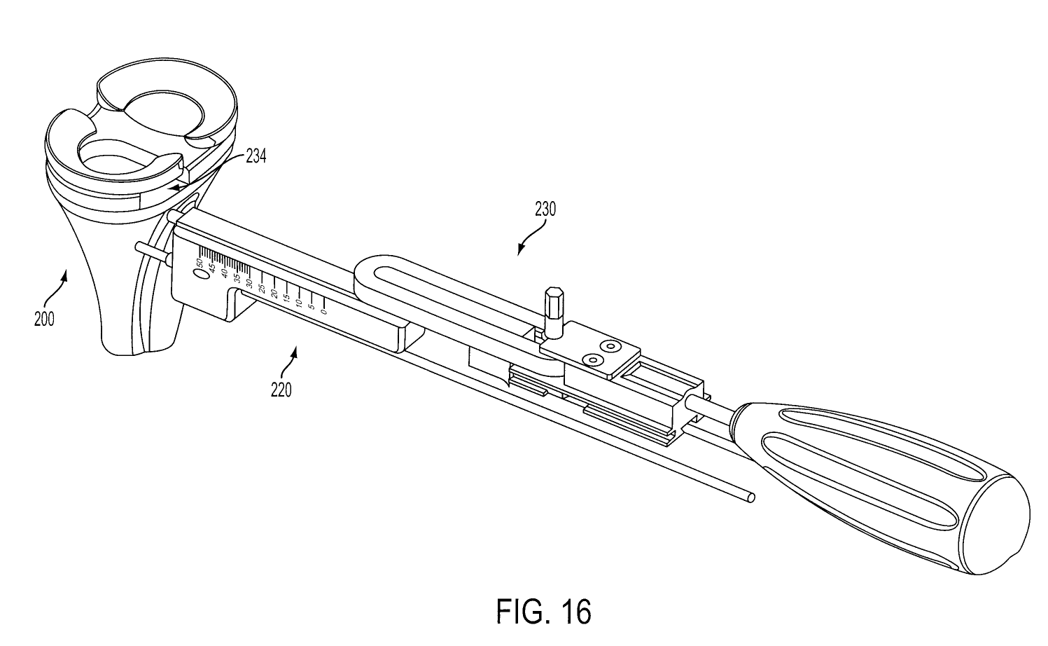

FIGS. 9 through 17 depict yet another exemplary embodiment of a resection system consistent with the present disclosure. Turning first to FIGS. 9 through 11, according to one aspect of this embodiment, a cutting path may be established relative to an anatomical feature to be resected, e.g., a portion of a tibia 200. An aiming device 202 may be located relative to the tibia 200. The aiming device 202 may include an aiming portion 204, which may be located relative to, or contact, the tibia 200, e.g., the tibial articular surface. For example, the aiming portion 204 may contact a portion of the articular surface of the tibia 200 to be removed during the resection. The aiming device 202 may further include an alignment portion, such as alignment portion 206. The alignment portion 206 may cooperate with a locating device 208 for establishing one or more axes relative to the tibia 200. The aiming device 202 may include indicia for indicating a position of the locating device 208 relative to the aiming device 202. In one embodiment, the alignment portion 206 may be received in a cooperating channel or trough 210 in the locating device 208. The aiming device 202 and the locating device 208 may be slidably coupled and releasably securable to one another to maintain a desired relationship between the aiming device 202 and the locating device 208 and relative to the tissue to be resected. In other embodiments, the aiming device 202 and the locating device 208 may comprise a single instrument, etc.

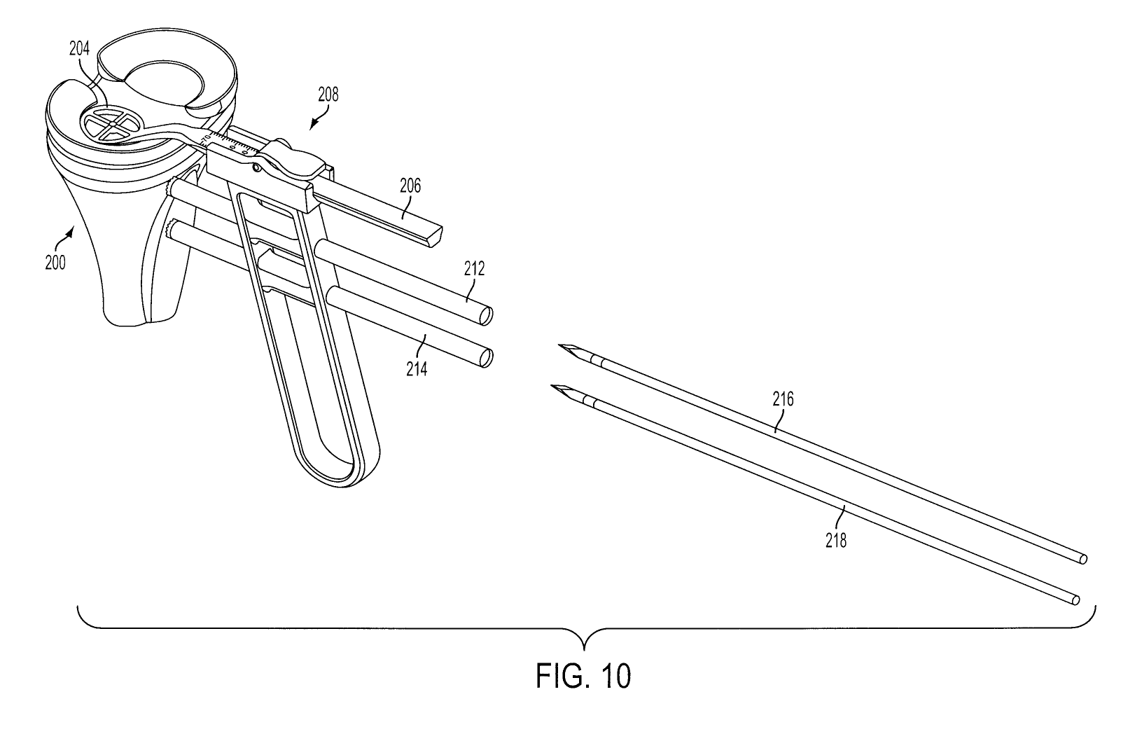

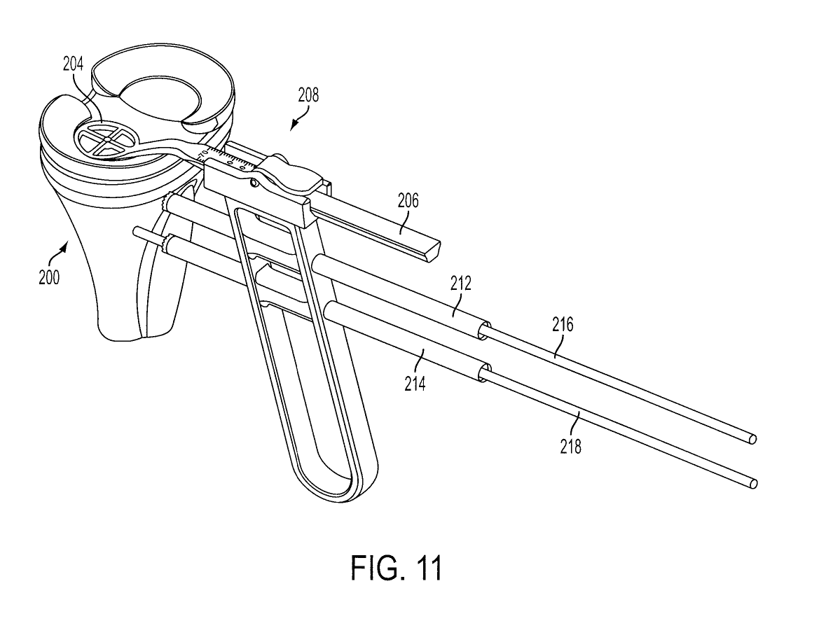

In addition to cooperating with the aiming device 202, the locating device 208 may include one or more elements for establishing working axes relative to the tibia 200. As shown, for example, in FIGS. 10 and 11, the locating device 208 may include at least one aperture, e.g., cannulated member 212 and/or 214, oriented along respective working axes. At least one guide pin 216 and/or 218 may be at least partially received through the cannulated member 212 and/or 214 and may engage the tibia 200. In this way, the locating device 208 may orient the one or more guide pins 216, 218 relative to the tissue to be resected. For example, the at least one guide pin 216 and/or 218 may be drilled into, or otherwise inserted, extending at least partially into the tibia 200. The at least one guide pin 216 and/or 218 may establish a trajectory and an orientation relative to the tibia 200. Once the at least one guide pin 216 and/or 218 has engaged the tibia 200 to define respective axes, the aiming device 202 and the locating device 208 may be removed.

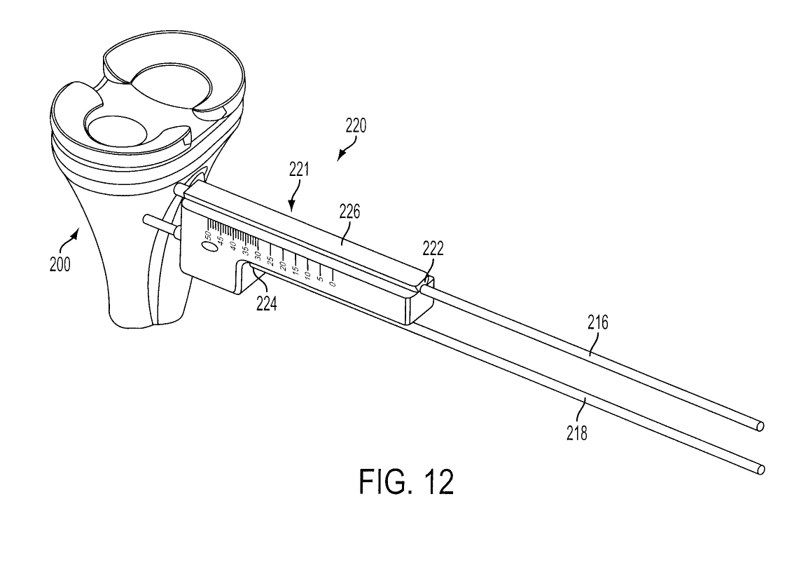

Turning to FIGS. 12 through 16, the axes provided by the at least one guide pin 216 and/or 218 may be used to guide the resection of a portion of the tibia 200. FIG. 12 depicts a cutting guide 220 according to this embodiment. The cutting guide 220 of this embodiment includes a guide device 221 and at least one guide pin 216 and/or 218. The at least one guide pin 216 and/or 218 may be secured to at least one anatomical feature to define an axis relative to tissue to be resected. The guide device 221 may cooperate with the at least one guide pin 216 and/or 218 and may assume an alignment and orientation based on the axes of the at least one guide pin 216 and/or 218. For example, the guide device 221 may be slidably coupled to the at least one guide pin 216 and/or 218 and may position the guide device 221 relative to the tissue to be resected along an axis defined by the at least one guide pin 216 and/or 218. For example, the guide device 221 may include apertures 222, 224 configured to at least partially receive the at least one guide pin 216 and/or 218 therethrough.

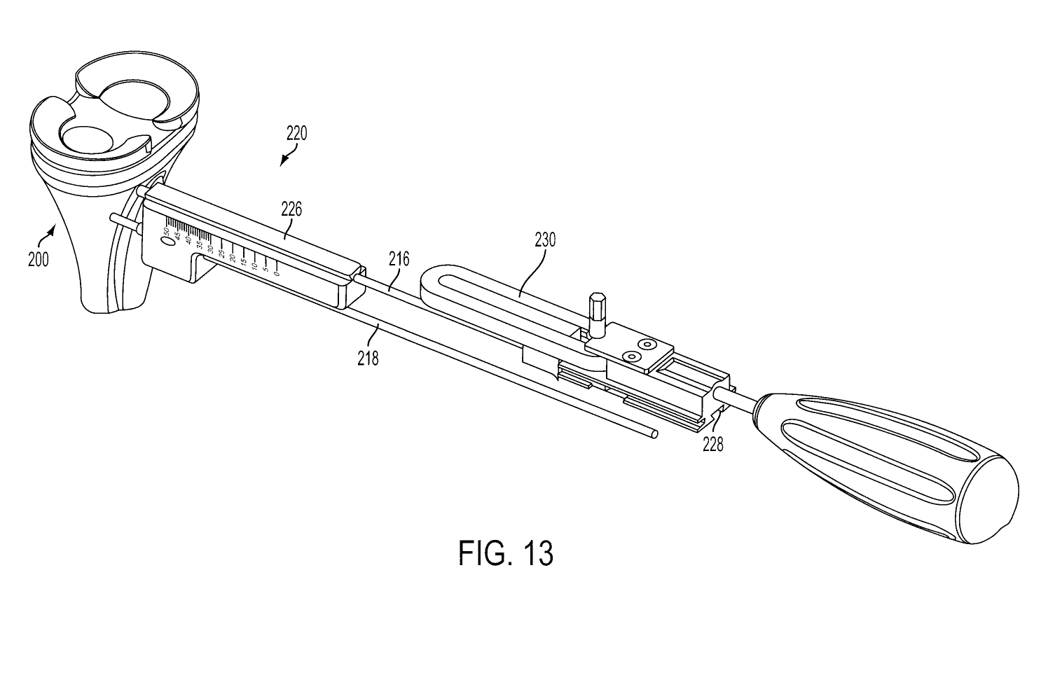

The guide device 221 may be slidably coupled to a cutter 230 to position the cutter 230 relative to tissue to be resected. The guide device 221 may include a guide portion, such as a dovetail rail 226. The dovetail rail 226 may constrain the movement of the cutter 230 along the axis defined by the at least one guide pin 216 and/or 218. Various other guide portions may also be employed in connection with the present disclosure, such as ribs, rails, rods, beaded ribs, grooves, etc. Referring briefly to FIGS. 13-16, a cooperating guide portion on a cutter 230, such as a complementary dovetail groove 228, or other portion capable of cooperating with the guide portion of the guide device, may interact with the dovetail rail 226 to guide the movement of the cutter 230 relative to the cutting guide 220. The cutter 230 shown in FIGS. 13-16 is a representational depiction which does not show any particular cutters and drive mechanisms. Suitable cutters and drive mechanisms may be consistent with the previously described cutter, e.g., shown in FIGS. 1, and 3-7. Various other embodiments of a cutter may also suitably be employed in connection with the present disclosure.

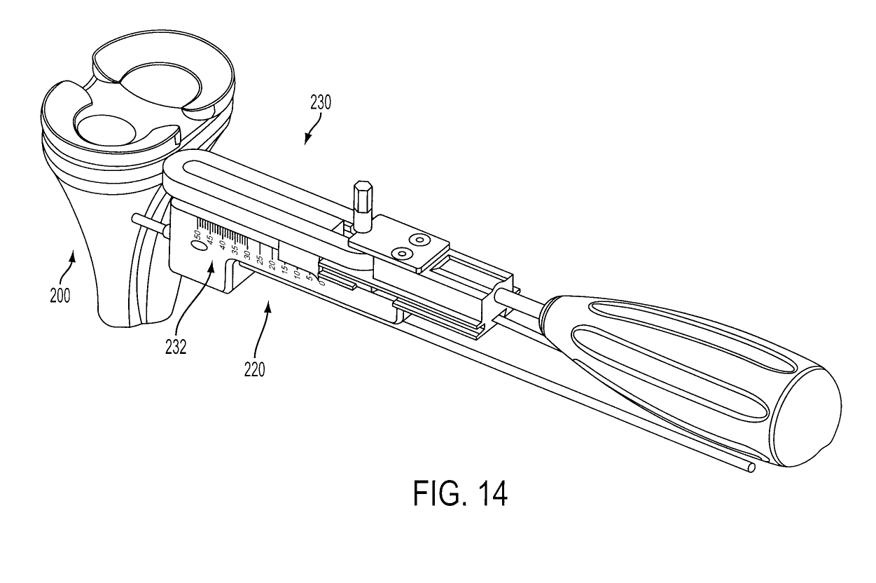

As shown, for example, in FIGS. 14 through 16, the cutter 230 may be advanced into the tibia 200 along a path defined by, at least in part, the dovetail rail 226. Consistent with the disclosure relative to FIGS. 1, and 3-7, the cutter 230 may include a blade system for resecting material as the cutter 230 may be advanced into the tibia 200. The depth of the resection may be controlled, at least in part, by the cutting guide 220. In one embodiment, and referring specifically to FIG. 14, the guide device 221 may include indicia 232 which may indicate position, e.g., depth of advancement of the cutter 230, relative to the guide device 221. Movement of the cutter 230 relative to the indicia 232 may facilitate controlling the depth of advancement of the cutter 230 into the tibia 200. In other embodiments, stops may be associated with the cutter and/or with the guide device 221 to limit the range of advancement of the cutter 230 relative to the guide device 221, and therein relative to the tibia, in order to control the depth of resection.

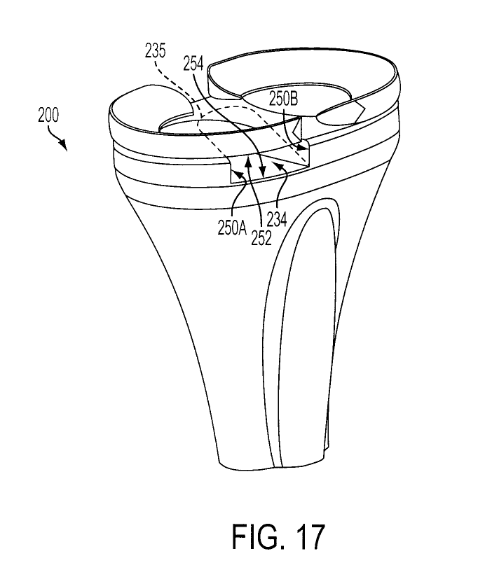

As depicted, for example, in FIG. 17, and consistent with the foregoing description, advancing the cutter 230 into, and withdrawing the cutter 230 from, the tibia, as shown in FIGS. 14-16, may provide a resected pocket 234 or slot in the tibia 200. The shape of the pocket 234 may be based on, at least in part, the geometry of the cutter 230, the path of travel of the cutter 230, as controlled by the guide device 221 and the arrangement of the guide pins 216, 218. For example, in the illustrated embodiment, the cutter 230 may include a rounded distal end, which may produce a radiused interior end 235 of the pocket 234. Alternatively or additionally, the shape of the cutting portion of the links of the blade system (as depicted in FIG. 1, for example) may be selected to generate a desired geometry of the pocket 234. In other embodiments the cutter 230 may have a flatter, or squared-off, distal end, producing a corresponding pocket geometry. Additionally, more than one resection cut may be carried out to create various pocket geometries, such as undercuts, dovetails, eccentric pockets, etc. Further, while the embodiment described herein depict the cutting guide as directly attached to a anatomical structure (via, for example, screws or guide pins) it is equally contemplated herein that the cutting guide may be attached to the anatomical structure in a less-invasive manner, for example, using straps or brackets that may stabilize the position of the cutting guide with respect to the tissue to be resected.

The cutter described in any of the embodiments herein may generally have a thickness that is greater than a conventional tissue cutting instrument. Thus, the pocket 234 generated by the cutter described herein may include defined sidewall surfaces 250A and 250B. The sidewall surfaces 250A and/or 250B may be generally normal to the top and bottom surfaces 252 and 254, respectively. Advantageously, the pocket 234 including sidewall surfaces 250A and/or 2150B generated by the cutter described herein may have sufficient height to accommodate a variety of implants without the need for additional cutting.

Advantageously, consistent with the present disclosure, it may, therefore, be possible create a clean and precise cavity, e.g., for receiving an implant. For example, in at least one embodiment described herein, a cutter is slidably coupled to a cutting guide. The cutting guide may be This The controllability of the size and shape of the resected slot may, in some instances, lend itself to the use of standard size implants. The controllability of the cutting system may also allow the resection of a desired area and volume of material using a minimally invasive procedure along a single approach. Additionally, the system herein may have lower demands of dexterity and experience to create the resected site than may be required using convention freehand techniques.

Various elements, aspects, and embodiments have been described herein. The elements, aspects, and embodiments are susceptible to combination with one another as well as to variation and modification, as will be understood by those having skill in the art. The present disclosure should, therefore, be considered to encompass such combinations, variations and modifications.

What is claimed is:

1. A tissue resection system, comprising:
a cutter comprising a blade system and a guide bar, said blade system is configured to be slidably coupled to said guide bar to permit said blade system to move relative to said guide bar, wherein said blade system comprises a chain of a plurality of links hingedly coupled together, at least one said link comprising a cutting portion to cut tissue, said blade system having a thickness configured to generate a recessed pocket in the tissue.

2. The system of claim 1, wherein said recessed pocket having sidewall surfaces a top and a bottom surface, said sidewall surfaces are substantially normal to said top and bottom surfaces.

3. The system of claim 1, wherein the overall shape of said recessed pocket is based on, at least in part, the shape of the cutter and the path of travel of the cutter.

4. The system of claim 1, wherein the overall shape of said recessed pocket is based on, at least in part, the geometry of the cutting portion of the at least one link of the blade system.

5. The system of claim 1, wherein said guide bar includes a slot formed therein; and said tissue resection system further comprises:
a cutting guide comprising a longitudinal member and a locating member disposed at an angle relative to said longitudinal member; said locating member is configured to orient the longitudinal member with respect to tissue to be resected; said longitudinal member includes at least one boss extending therefrom, said boss is configured to mate with said slot to slidably couple said guide bar to said longitudinal member to position said cutter relative to said tissue.

6. The system of claim 5, wherein said cutter further comprises a spring disposed in said slot, said spring is configured to bias said cutter toward a retracted position away from said tissue.

7. The system of claim 1, further comprising a drill configured to drive the blade system to travel around the guide bar.

8. The system of claim 5, said cutting guide further comprising at least one screw coupled to said locating member and configured to removably affix the cutting guide relative to said tissue.

9. The system of claim 1, wherein said guide bar further comprises a protruding rib disposed around at least a portion of the periphery of said guide bar, and said blade system comprises a groove for receiving said protruding rib.

10. The system of claim 1, further comprising:
a base member, said cutter is coupled to said base member; and
a cutting guide comprising a longitudinal member and a locating member disposed at an angle relative to said longitudinal member; said locating member is configured to orient the longitudinal member with respect to tissue to be resected; said longitudinal member is configured to be slidably coupled to said cutter to position said bases member relative to said tissue.

11. The system of claim 10, wherein said base member further comprises a slot; said slot is configured to receive said longitudinal member therein.

12. The system of claim 1, wherein said cutter further comprises a handle; said handle is coupled to said guide bar to facilitate movement of said cutter.

13. The system of claim 1, further comprising:
a cutting guide comprising a guide device and at least one guide pin, said guide pin is configured to be secured to at least one anatomical feature to define an axis relative to tissue to be resected; said guide device is configured to be slidably coupled to said guide pin to position said guide device relative to said tissue to be resected along said axis defined by said guide pin, said guide device is further configured to be slidably coupled to said cutter to position said cutter relative to said tissue.

14. The system of claim 13, further comprising: an aiming device and a locating device, said aiming device is configured to be positioned relative to said tissue to be resected; said locating device is configured to be slidably coupled to said aiming device to position said locating device relative to said tissue to be resected, said locating device is further configured to receive said at least one guide pin to orient said guide pin relative to said tissue.

15. The system of claim 14, wherein said aiming device further comprises indicia for indicating a position of said locating device relative to said aiming device.

16. The system of claim 13, wherein said guide device further comprises indicia for indicating a position of said cutter relative to said guide device.

17. The system of claim 14, wherein said aiming device is configured to be removably positioned relative to said tissue.

18. The system of claim 13, wherein said guide device further comprises a dovetail rail configured to constrain the movement of said cutter along said axis defined by said at least one guide pin.

19. The system of claim 13, wherein said guide device further comprises at least one aperture configured to receive said at least one guide pin.

20. A tissue resection system, comprising:
a cutter comprising a blade system and a guide bar, said blade system is configured to be slidably coupled to said guide bar to permit said blade system to move relative to said guide bar, wherein said blade system comprises a chain of a plurality of links hingedly coupled together, at least one said link comprising a cutting portion to cut said tissue to be resected, said blade system having a thickness configured to generate a recessed pocket in tissue; and
a cutting guide configured to be removably secured relative to an anatomical feature, wherein said cutter is further configured to be slidably coupled to said cutting guide to orient the cutter with respect to the tissue and to control the depth of resection into the tissue.

21. The system of claim 20, wherein said recessed pocket having sidewall surfaces a top and a bottom surface, said sidewall surfaces are substantially normal to said top and bottom surfaces.

22. The system of claim 20, wherein the overall shape of said recessed pocket is based on, at least in part, the shape of the cutter and the path of travel of the cutter.

23. The system of claim 20, wherein the overall shape of said recessed pocket is based on, at least in part, the geometry of the cutting portion of the at least on link of the blade system.
